(19) United States
(12) Patent Application Publication
(10) Pub. No.: US 2013/0259905 A1
(43) Pub. Date: Oct. 3, 2013

(54) COMPOSITION CAPABLE OF INDUCING ENDOPLASMIC RETICULUM STRESS

(71) Applicants: Chen Han-Min, Taipei (TW); Tan Shan-Wen, Taipei (TW)

(72) Inventors: Chen Han-Min, Taipei (TW); Tan Shan-Wen, Taipei (TW)

(73) Assignee: GOLD NANOTECH INC., Taipei (TW)

(21) Appl. No.: 13/541,948

(22) Filed: Jul. 5, 2012

(30) Foreign Application Priority Data
Mar. 28, 2012 (TW) 101110875

(51) Int. Cl. A61K 33/24; A61P 35/00; A61K 9/14; A61P 35/02; B82Y 5/00; B82B 1/00

(52) U.S. Cl. 424/400; 424/649; 977/773; 977/810; 977/915

(57) ABSTRACT

A composition capable of inducing endoplasmic reticulum stress including nanogold particles and a solvent is disclosed. After a chronic myelogenous leukemia patient intakes the composition capable of inducing endoplasmic reticulum stress, the endoplasmic reticulum stress of the chronic myelogenous leukemia cells is induced to cause apoptosis and cell death, so as to alleviate and control chronic myelogenous leukemia.

### BACKGROUND OF THE INVENTION

1. Field of the Invention

The present invention relates to a composition capable of inducing endoplasmic reticulum stress, and more particularly to the composition capable of inducing endoplasmic reticulum stress of human cells.

2. Description of Related Art

Chronic myelogenous leukemia is one of the myeloproliferative disorders, and some chronic myelogenous leukemia patients may not have significant symptoms at early stage, until an abnormal increase of leukocytes is found and diagnosed during a physical examination.

The chronic myelogenous leukemia generally relates to an abnormal translocation of chromosome. In such translocation, BCR genes in the 22^{nd} pair of chromosomes in human body and ABL genes in the 9^{th} pair of chromosomes are translocated and then fused together. As a result, the abnormally fused genes produce BCR-ABL proteins which inhibit chromosomal repair, cause genomic instability and genetic mutation, and form blood cancer cells.

Bone marrow transplantation was generally used for the treatment of chronic myelogenous leukemia, but most of the patients fail to pass the drug test, and thus the bone marrow transplantation is used at a declining rate now. Since apoptosis can cause cell death, apoptosis can be considered as another alternative for the treatment. Related reports show that excessive endoplasmic reticulum (ER) stress can induce apoptosis, and the excessive endoplasmic reticulum stress is primarily related to an unfolded protein response, which refers to the response of unfolded or misfolded proteins with an accumulation up to a certain quantity in the lumen of the endoplasmic reticulum.

Related articles indicated that the stronger the unfolded protein response, the better is the CHOP expression, and this result is closely related to the death of cells (Refer to Brewer J. W. et al., “A pathway distinct from the mammalian unfolded protein response regulates expression of endoplasmic reticulum chaperones in non-stressed cells.” EMBO J., 16:7207-7216 (1997)). Related articles further indicated that the apoptosis is related to a drop of Calnexin expression (Refer to Renee Guerin et al., “Calnexin Is Involved in Apoptosis Induced by Endoplasmic Reticulum Stress in the Fission Yeast” PMC J., 19(10):4404-4420 (2008)). In addition, articles also disclosed that endoplasmic reticulum stress will expand the activity of PERK (Refer to Harding HP. et al., “Protein translation and folding are coupled by an endoplasmic-reticulum-resident kinase” PMC J., 397(6716):271-4 (1999)).

CHOP is a basic leucine zipper (bZIP) transcription factor of a C/EBP family, and will be induced at an early stage of the endoplasmic reticulum stress, and thus CHOP is often used as the best choice for verifying the endoplasmic reticulum stress response in mammalian cells. Calnexin is a 90 kDa integral protein on the endoplasmic reticulum and a chaperone molecule having the properties of assisting the endoplasmic reticulum to perform protein folding and quality control, and assuring that only the properly folded and assembled proteins can enter or exit the endoplasmic reticulum along a secretory pathway. In addition, calnexin retains the unfolded or unassembled N-linked glycoproteins in the endoplasmic reticulum. Protein kinase-like endoplasmic reticulum kinase (PERK) is an eukaryotic initiation factor 2α (eIF2α) kinase of a phosphorylation, and exists in form of a transmembrane protein in the endoplasmic reticulum, so that PERK is related to the endoplasmic reticulum stress. If the endoplasmic reticulum stress has an abnormal translation, signals transmitted by the endoplasmic reticulum stress will be responded as well, wherein the abnormal translation of the endoplasmic reticulum stress will expand the activity of the PERK such as phosphorylation, and such activity refers to the activity of using the eIF2α kinase of the phosphorylation or PERK to show the phenomenon of a reduced endoplasmic reticulum translation.

### SUMMARY OF THE INVENTION

In view of the aforementioned problems of the prior art, it is an objective of the present invention to provide a composition capable of inducing endoplasmic reticulum stress, such that after a chronic myelogenous leukemia patient intakes the composition capable of inducing endoplasmic reticulum stress, an endoplasmic reticulum stress of the chronic myelogenous leukemia is induced to cause apoptosis and cell death, so as to alleviate and control the medical conditions of chronic myelogenous leukemia.

To achieve the aforementioned objective, the present invention provides a composition capable of inducing endoplasmic reticulum stress comprising a plurality of nanogold particles and a solvent, wherein one gram of the composition has a content of nanogold particles ranging from 0.5 μg to 5 μg; the nanogold particles have a particle size ranging from 1 nm to 30 nm; and the composition capable of inducing endoplasmic reticulum stress can induce an endoplasmic reticulum stress of human chronic myelogenous leukemia K562 cells.

Another objective of the present invention is to provide a composition capable of inducing endoplasmic reticulum stress, such that after a lymphoma patient intakes the composition capable of inducing endoplasmic reticulum stress, an endoplasmic reticulum stress of the lymphoma is induced to cause apoptosis and cell death, so as to alleviate and control the medical conditions of lymphoma.

To achieve the aforementioned objective, the present invention provides a composition capable of inducing endoplasmic reticulum stress, comprising a plurality of nanogold particles and a solvent, wherein each gram of the composition has a content of nanogold particles ranging from 0.5 μg to 5 μg; the nanogold particles have a particle size ranging from 1 nm to 30 nm; and the composition capable of inducing endoplasmic reticulum stress can induce an endoplasmic reticulum stress of human lymphoma cells.

### BRIEF DESCRIPTION OF THE DRAWINGS

FIG. 1A is a CHOP expression chart of 5-nm nanogold particles at different dosages;

FIG. 1B is a calnexin expression chart of 5-nm nanogold particles at different dosages;

FIG. 1C is a PERK phosphorylation chart of 5-nm nanogold particles at different dosages;

FIG. 2A is a CHOP expression chart of 5-nm nanogold particles in various testing cells;

FIG. 2B is a calnexin expression chart of 5-nm nanogold particles in various testing cells;

FIG. 2C is a PERK phosphorylation chart of 5-nm nanogold particles in various testing cells;

FIG. 3A is a CHOP expression chart of different sized nanogold particles at the dosage of 0.5 ppm;

FIG. 3B is a CHOP expression chart of different sized nanogold particles at the dosage of 5 ppm;

FIG. 3C is a calnexin expression chart of different sized nanogold particles at the dosage of 0.5 ppm;

FIG. 3D is a calnexin expression chart of different sized nanogold particles at the dosage of 5 ppm;

FIG. 3E is a PERK phosphorylation chart of different sized nanogold particles at the dosage of 0.5 ppm;

FIG. 3F is a PERK phosphorylation chart of different sized nanogold particles at the dosage of 5 ppm;

FIG. 4A shows the expression of the capase 3 after K562 cells are reacted with different compositions for 48 hours;

FIG. 4B shows the expression of capase 3 after K562 cells are reacted with nanogold particles within 48 hours, including the time after 0 hour, 12 hours, 24 hours and 48 hours;

FIG. 5 shows the test results after 200 nM of endoplasmic reticulum thapsigargin (TG) and nanogold particles of 5 ppm are reacted with K562 cells and an immunostaining method is used for performing the test for 0 hour, 3 hours, 6 hours, 12 hours, 24 hours and 48 hours;

Table 1 lists the test results of 5-nm nanogold particles at different dosages;

Table 2 lists the test results of 5-nm nanogold particles in four different testing cells;

Table 3 lists the test results of different sized nanogold particles at the dosages of 0.5 ppm and 5 ppm; and

Table 4 lists the expression of different proteins after K562 cells are reacted with nanogold particles.

### DETAILED DESCRIPTION OF THE PREFERRED EMBODIMENTS

The technical characteristics of the present invention will become apparent with the detailed description of preferred embodiments and the illustration of related drawings as follows.

Small nanogold particles (AuNPs) have special properties including electric property, biocompatibility and molecular recognizability, such that the use of nanogold particles in molecular biology is discussed extensively. Since gold based compositions are proven as compositions with medical treatment effects and have been used for different diseases such as the treatment for rheumatoid arthritis, cancer, AIDS, bronchial asthma and malaria, etc. and nanogold particles also have potentials for curing cancers, therefore it is absolutely necessary to evaluate the cytotoxicity of nanogold particles and the molecular and physical effects induced by nanogold particles.

Endoplasmic reticulum (ER) stress is one of the molecular physical phenomena and there are three main responses to the endoplasmic reticulum stress including (1) endoplasmic reticulum (ER) associated degradation (2) Unfolded protein response and (3) apoptosis, wherein apoptosis generally results in cell death. In summation of the description above, endoplasmic reticulum stress can induce cell death.

The testing cells are K562 cells, which are the first human immortalized myeloid leukemia cell line, and are used extensively in reference data of many cell lines since 1970. The K562 cells can be differentiated by the simulation of different inducers in vitro, so that the K562 cells are considered as a very useful typical model, not only having the capability of testing the potential of medical treatment of a new composition, but also being used in studying the expression of molecules in embryos and fatal human protein genes as well as their varying process.

Since the nanogold particles can induce a molecular physical phenomenon such as the endoplasmic reticulum stress, therefore the inventor of the present invention conducted experiments to show that nanogold particles can induce the of endoplasmic reticulum stress of the K562 cells, and further can induce the cell death.

The occurrence of endoplasmic reticulum stress can be confirmed by any of the following phenomena: an increase of CHOP expression, a decrease of calnexin expression, and an increase of PERK phosphorylation. To observe the aforementioned phenomena, the nanogold particles are reacted with the cells for at least 24 hours, and an immunostaining method is used for the observation. The experimental procedure of the present invention comprises the following steps:

(I) All nanogold particles are dissolved in distilled water.

(II) The distilled water is introduced to the testing cells and reacted with the testing cells according to the experiment requirements.

(III) Different expressions and phosphorylation responses of each test cellular protein are standardized by a control, wherein the control has a value set to 100%.

(IV) Image analysis software (TotalLab 120, Nonlinear) is provided for measuring the protein response observed in the immunostaining method.

In addition, the aforementioned experiments are repeated for three times to obtain the average values in order to achieve the best data. Any of the following indicates the occurrence of an endoplasmic reticulum stress of the cells:

(1) CHOP expression>100%

(2) 0%≦Calnexin expression<100%

(3) PERK phosphorylation>100%

First Experiment Results

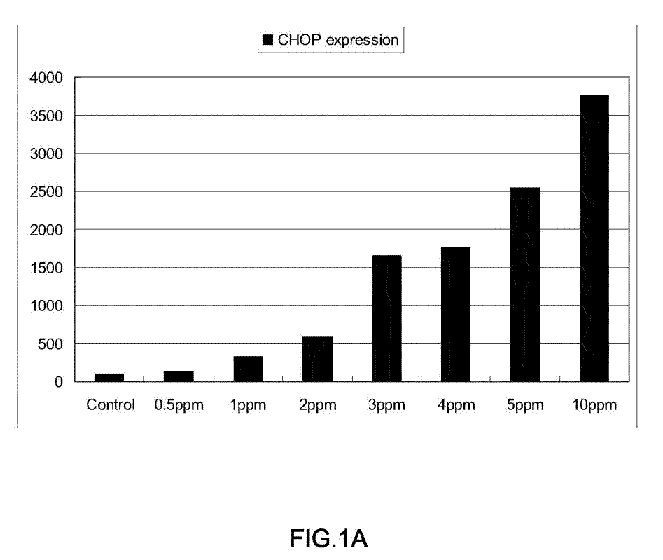

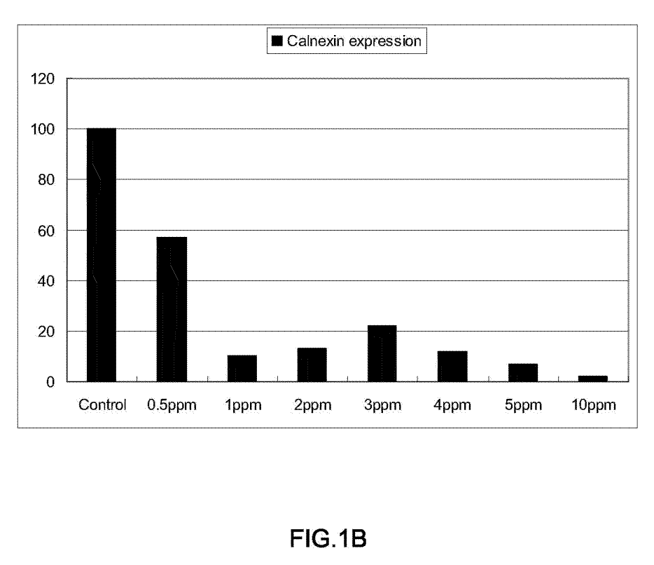

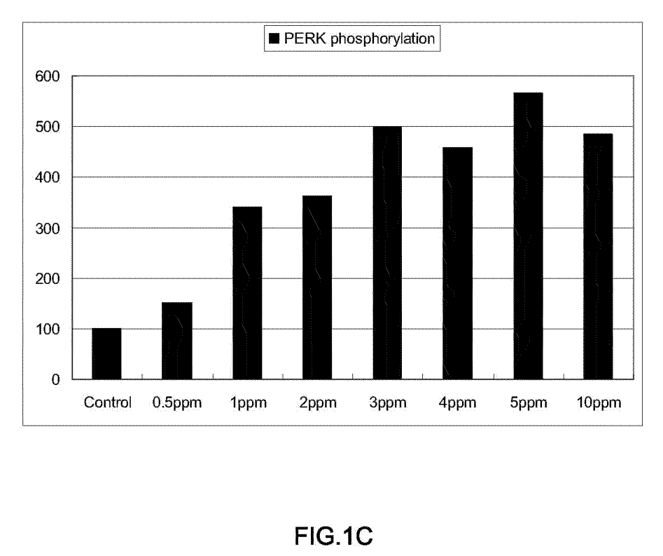

The present invention discloses a composition capable of inducing endoplasmic reticulum stress, comprising a plurality of nanogold particles (AuNPs) and a solvent, wherein the solvent can be distilled water. With reference to Table 1 for the test results of 5-nm nanogold particles at different dosages and FIGS. 1A to 1C for a CHOP expression chart, a calnexin expression chart and a PERK phosphorylation chart of 5-nm nanogold particles at different dosages respectively, Table 1 shows the test result of K562 cells reacted with 5-nm nanogold particles, and if the dosage of the 5-nm nanogold particles is 0.5 ppm, the CHOP and calnexin expressions are 120%±5 and 57%±3 respectively, so that if the dosage of the 5-nm nanogold particles is 0.5 ppm, then the endoplasmic reticulum stress of the K562 cells will be induced. As the dosage increases, the response of the endoplasmic reticulum stress becomes more significant. If the dosage of the 5-nm nanogold particles is 0.5 ppm, then the PERK phosphorylation will be 150%±7, so that if the dosage of the 5-nm nanogold particles is 0.5 ppm, then the endoplasmic reticulum stress of the K562 cells will be induced. For dosages equal to or greater than 3 ppm, the PERK phosphorylation will remain at the neighborhood of 500% and will not continue increasing.

TABLE 1

| | CHOP | Calnexin | PERK |
|---|---|---|---|
| | expression | expression | phosphorylation |
| Control | 100 | 100 | 100 |

| | | | | |
|---|---|---|---|---|
| 0.5 | ppm | 120 ± 5 | 57 ± 3 | 150 ± 7 |
| 1 | ppm | 330 ± 10 | 10 ± 4 | 339 ± 18 |
| 2 | ppm | 578 ± 19 | 13 ± 2 | 362 ± 42 |
| 3 | ppm | 1655 ± 49 | 22 ± 3 | 498 ± 10 |
| 4 | ppm | 1752 ± 35 | 12 ± 1 | 458 ± 32 |
| 5 | ppm | 2542 ± 39 | 7 ± 3 | 565 ± 44 |
| 10 | ppm | 3756 ± 156 | 2 ± 1 | 485 ± 13 |

Second Experiment Results

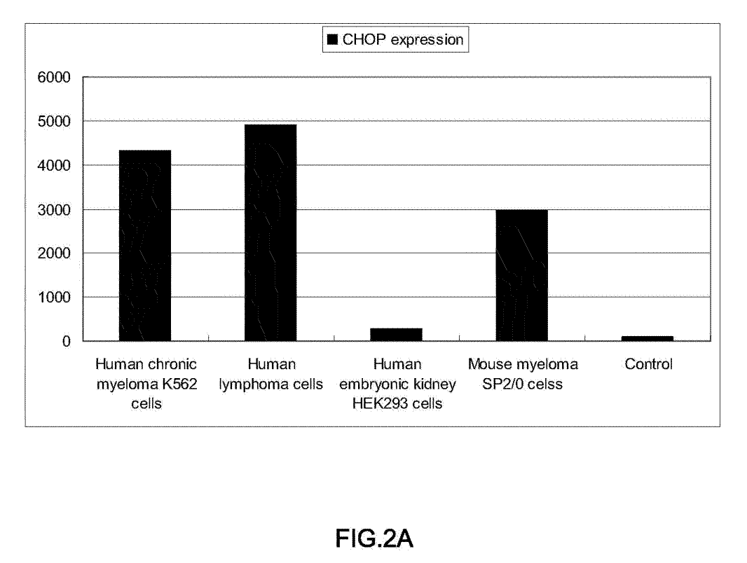

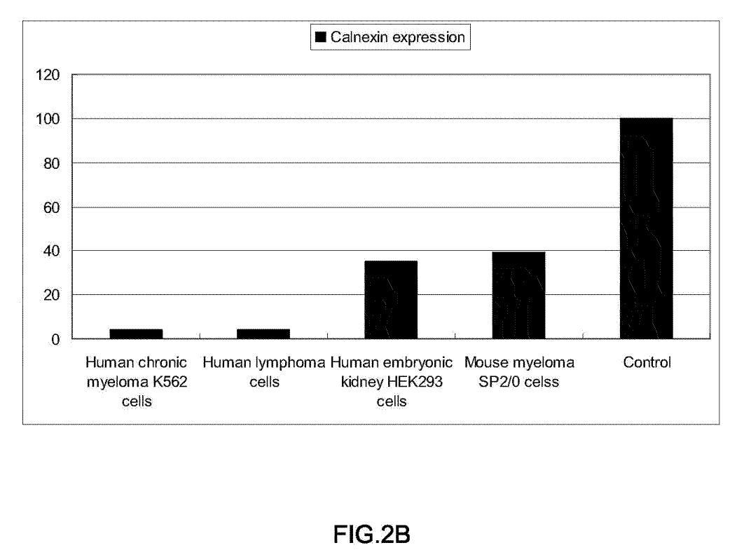

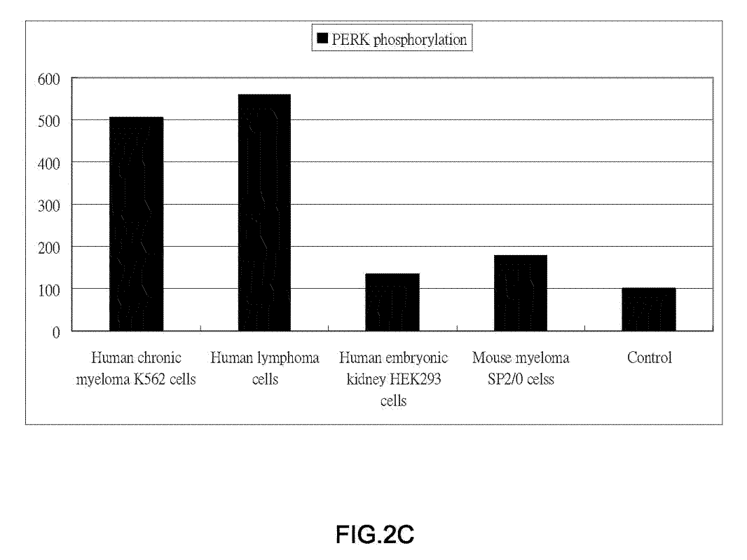

Table 2 lists the test results of 5-nm nanogold particles in four different testing cells, and FIGS. 2A to 2C show a CHOP expression chart, a calnexin expression chart and a PERK phosphorylation chart of 5-nm nanogold particles in various testing cells respectively, wherein Table 2 shows the result of the K562 cells reacted with the 5-nm nanogold particles at the dosage of 5 ppm.

Table 2 indicates that if the CHOP expression of the K562 cells is 4322%±13, or the calnexin expression of the K562 cells is 4%±2, the CHOP expression of the human embryonic kidney (HEK293) cells will be 288%±18. In other words, the human embryonic kidney cells have a slight endoplasmic reticulum stress only. The inventor of the present invention observed that when slight endoplasmic reticulum stress of the human embryonic kidney cells is induced, apoptosis seldom occurs. After a period of time, the endoplasmic reticulum associated degradation reduces the endoplasmic reticulum stress. Therefore, if the CHOP expression of the K562 cells is 4322%±13, or the calnexin expression of the K562 cells is 4%±2, then the human embryonic kidney cells still fall within a safety range.

The composition of the present invention composition preferably contains 0.5 μg to 5 μg of nanogold particles per gram of the composition.

TABLE 2

| | CHOP | Calnexin | PERK |
|---|---|---|---|
| | expression | expression | phosphorylation |
| Human chronic | 4322 ± 13 | 4 ± 2 | 506 ± 13 |
| myeloma K562 | | | |
| cells | | | |
| Human | 4912 ± 236 | 4 ± 2 | 559 ± 39 |
| lymphoma cells | | | |
| Human | 288 ± 18 | 35 ± 7 | 135 ± 10 |
| embryonic | | | |
| kidney HEK293 | | | |
| cells | | | |
| Mouse myeloma | 2955 ± 152 | 39 ± 1 | 177 ± 12 |
| SP2/0 cells | | | |
| Control | 100 | 100 | 100 |

Third Experiment Results

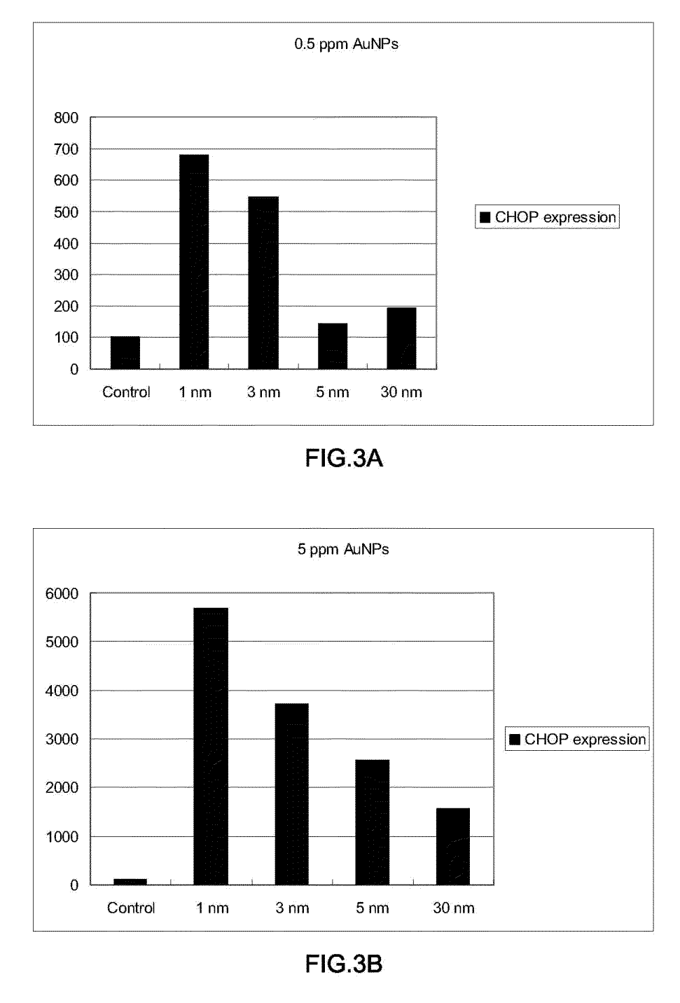

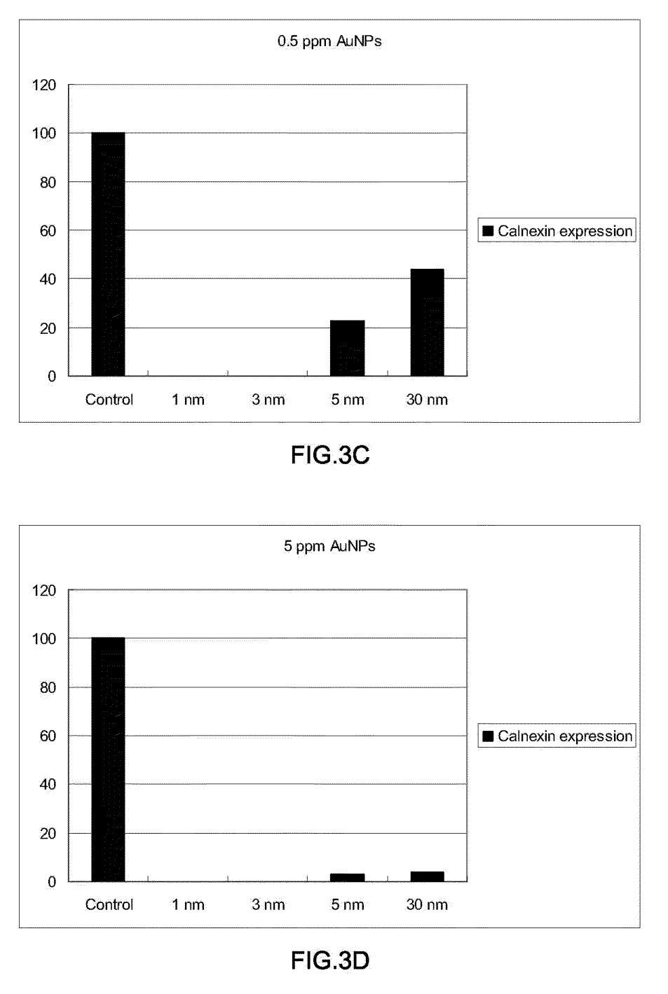

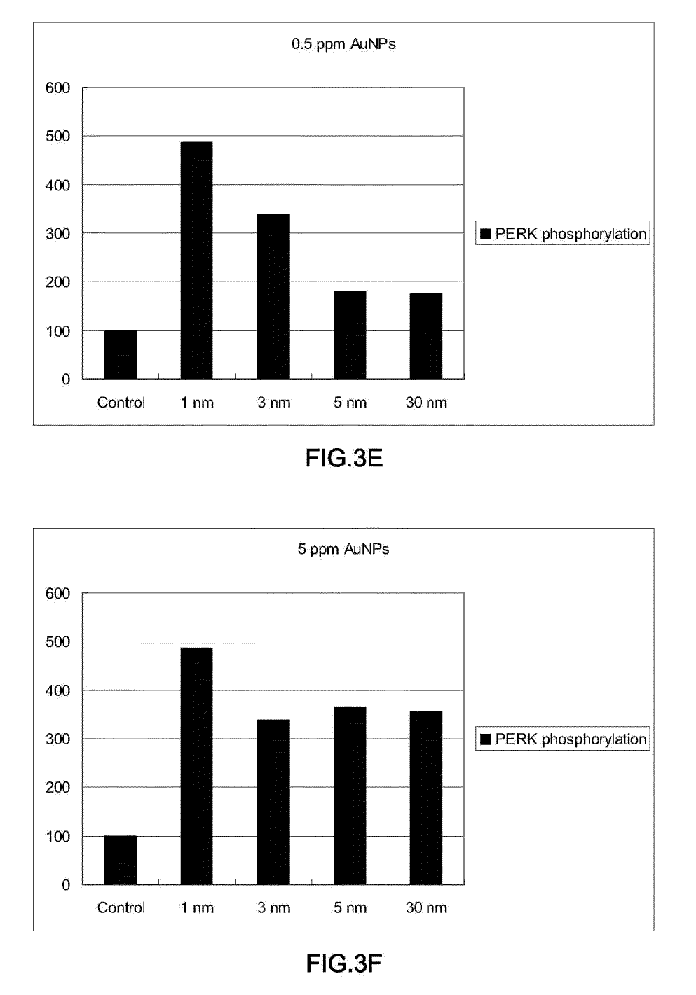

Table 3 lists the test results of different sized nanogold particles at the dosages of 0.5 ppm and 5 ppm, and FIGS. 3A-3F show the CHOP expression charts, the calnexin expression charts and the PERK phosphorylation charts of different sized nanogold particles at the dosages of 0.5 ppm and 5 ppm respectively, and Table 3 indicates that the size of nanogold particles preferably falls with a range from 1 nanometer (nm) to 30 nm.

TABLE 3

| | | |
|---|---|---|
| CHOP | Calnexin | PERK |
| expression | expression | phosphorylation |

0.5 ppm AuNPs

| | | | |
|---|---|---|---|
| Control | 100 | 100 | 100 |
| 1 nm | 678 ± 26 | 0 | 486 ± 12 |
| 3 nm | 544 ± 34 | 0 | 339 ± 17 |
| 5 nm | 144 ± 7 | 23 ± 3 | 180 ± 17 |
| 30 nm | 194 ± 25 | 44 ± 2 | 175 ± 9 |

5 ppm AuNPs

| | | | |
|---|---|---|---|
| Control | 100 | 100 | 100 |
| 1 nm | 5682 ± 579 | 0 | 486 ± 12 |
| 3 nm | 3712 ± 144 | 0 | 339 ± 17 |
| 5 nm | 2546 ± 159 | 3 ± 1 | 366 ± 24 |
| 30 nm | 1564 ± 45 | 4 ± 2 | 355 ± 59 |

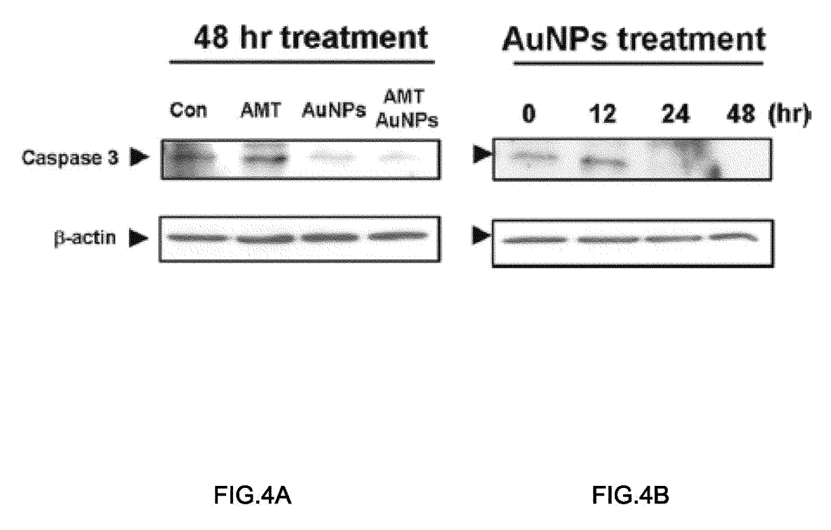

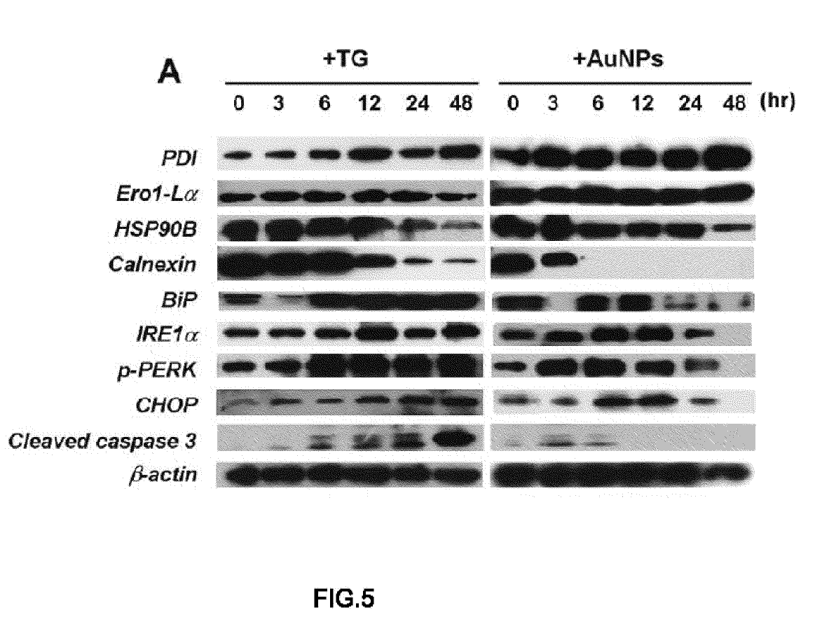

With reference to FIGS. 4A and 4B for the expression of caspase 3 after the K562 cells are reacted with different compositions for 48 hours, and the expression of caspase 3 after the K562 cells are reacted with the nanogold particles within 48 hours, including the time after 0 hour, 12 hour, 24 hours and 48 hours respectively, β-actin is used as a control, and AMT refers to aminopterin which is an amino folic acid mainly used for the treatment of acute leukemia. FIGS. 4A and 4B further confirm that nanogold particles can induce apoptosis of the K562 cells.

With reference to FIG. 5 for the test results after 200 nM of endoplasmic reticulum thapsigargin (TG) and nanogold particles of 5 ppm are reacted with K562 cells and an immunostaining method is used for performing the test for 0 hour, 3 hours, 6 hours, 12 hours, 24 hours and 48 hours. PDI, Ero1-Lα, HSP90B, calnexin, BiP, IRE1α, phosphorylated PERK, CHOP, and cleaved caspase 3 are used in the test. The data shown in FIG. 5 indicates that the reaction of 5 ppm nanogold particles with K562 cells can provides a stronger apoptosis phenomenon than that induced by the reaction of 200 nM endoplasmic reticulum TG with K562 cells.

With reference to Table 4 for the expression of various different proteins after the K562 cells are reacted with the nanogold particles, proteins including G1-G15 not reacted with the nanogold particles show an up-regulated condition, and a total of eight types of proteins C1-C8 reacted with the nanogold particles show a down-regulated condition, and thus indicating that nanogold particles can induce endoplasmic reticulum stress to K562 cells. For example, heat shock protein has different responses in G1 and C1.

TABLE 4

| | | | Mᵣ/pI | | MOWSE | No. of peptides | | Sequence | Relative |
|---|---|---|---|---|---|---|---|---|---|
| Spot no. | Accession no. | Protein description | observed | theoretical | score | queried | matched | coverage (%) | expression ratio |
| C1 | gi\|292160 | Heat shock protein 70 | 97.9/5.10 | 78.9/5.13 | 39 | 41 | 1 | 1 | 0.01 |
| C2 | gi\|6005942 | Valosin-containing protein | 89.7/5.17 | 89.3/5.14 | 554 | 54 | 14 | 18 | 0.01 |
| C3-1 | gi\|292059 | MTHSP75 | 70.5/5.54 | 73.7/5.97 | 978 | 97 | 28 | 36 | 0.18 |
| C3-2 | gi\|62897075 | Heat shock 70 kDa | | 73.6/5.87 | 951 | | 27 | 36 | |
| | | protein 9B precursor | | | | | | | |
| C3-3 | gi\|7331218 | Keratin 1 | | 66.0/8.16 | 256 | | 5 | 7 | |
| C4-1 | gi\|7331218 | Keratin 1 | 47.8/5.80 | 66.0/8.16 | 95 | 44 | 2 | 4 | 0.01 |
| C4-2 | gi\|292059 | MTHSP75 | | 73.7/5.97 | 61 | | 1 | 2 | |
| C4-3 | gi\|5031753 | Heterogeneous nuclear | | 49.5/5.89 | 38 | | 1 | 4 | |
| | | ribonucleoprotein H1 | | | | | | | |
| C5 | gi\|7331218 | keratin 1 | 47.7/5.95 | 66.0/8.16 | 49 | 4 | 3 | 5 | 0.01 |
| C6-1 | gi\|306875 | C protein | 39.3/4.91 | 31.9/5.10 | 286 | 40 | 5 | 14 | 0.01 |
| C6-2 | gi\|193785255 | Unnamed protein product | | 32.3/4.99 | 271 | | 5 | 14 | |
| C6-3 | gi\|28317 | Unnamed protein product | | 59.5/5.17 | 122 | | 2 | 3 | |
| C6-4 | gi\|292059 | MTHSP75 | | 73.7/5.97 | 82 | | 1 | 2 | |
| C6-5 | gi\|386854 | Type II keratin subunit protein | | 52.8/5.31 | 42 | | 1 | 3 | |
| C7-1 | gi\|4506667 | Ribosomal protein P0 | 34.4/5.65 | 34.3/5.71 | 307 | 41 | 8 | 27 | 0.36 |
| C7-2 | gi\|189054178 | Unnamed protein product | | 66.0/7.62 | 118 | | 2 | 3 | |
| C8-1 | gi\|28317 | Unnamed protein product | 26.0/7.27 | 59.5/5.71 | 228 | 85 | 4 | 8 | 0.01 |
| C8-2 | gi\|189054178 | Unnamed protein product | | 66.0/7.62 | 155 | | 4 | 7 | |
| C8-3 | gi\|4139784 | Chain A, canine Gdp-Ran | | 57.9/8.04 | 84 | | 3 | 11 | |
| | | Q69I mutant | | | | | | | |
| G1-1 | gi\|306891 | 90 kDa heat shock protein | 86.7/5.21 | 83.2/4.97 | 375 | 61 | 8 | 11 | 7.15 |
| G1-2 | gi\|83318444 | HSP90AA1 protein | | 68.3/5.11 | 314 | | 8 | 12 | |
| G1-3 | gi\|189054178 | Unnamed protein product | | 66.0/7.62 | 118 | | 4 | 6 | |
| G1-4 | gi\|1082886 | TRAP-1 | | 75.3/8.43 | 95 | | 1 | 2 | |
| G2-1 | gi\|194388088 | Unnamed protein product | 70.5/4.90 | 63.9/5.39 | 269 | 132 | 7 | 12 | 6.35 |
| G2-2 | gi\|7331218 | Keratin 1 | | 66.0/8.16 | 265 | | 6 | 9 | |
| G2-3 | gi\|386785 | Heat shock protein | | 69.9/5.42 | 261 | | 7 | 12 | |
| G2-4 | gi\|35222 | Unnamed protein product | | 70.8/5.67 | 123 | | 3 | 4 | |
| G3-1 | gi\|340219 | Vimentin | 42.9/4.47 | 53.7/5.03 | 339 | 126 | 7 | 17 | 2.69 |
| G3-2 | gi\|189054178 | Unnamed protein product | | 66.0/7.62 | 210 | | 5 | 7 | |
| G3-3 | gi\|28336 | Mutant beta-actin (beta′-actin) | | 41.8/5.22 | 64 | | 1 | 4 | |
| G3-4 | gi\|307141 | Lysozyme precursor (EC 3.2.1.17) | | 16.5/9.38 | 57 | | 2 | 8 | |
| G3-5 | gi\|157835338 | Chain A, mutant human lysozymes | | 14.7/9.28 | 57 | | 2 | 9 | |
| G3-6 | gi\|157835340 | Chain A, human lysozyme | | 14.7/9.28 | 57 | | 2 | 9 | |
| G3-7 | gi\|113584 | Ig alpha-1 chain C region | | 37.6/6.08 | 39 | | 1 | 4 | |
| G4-1 | gi\|189054178 | Unnamed protein product | 36.6/5.00 | 66.0/7.62 | 156 | 114 | 3 | 5 | 7.30 |
| G4-2 | gi\|386785 | Heat shock protein | | 69.8/5.42 | 104 | | 2 | 3 | |
| G5-1 | gi\|189054178 | Unnamed protein product | 36.6/5.11 | 66.0/7.62 | 470 | 140 | 9 | 13 | 10.00 |
| G5-2 | gi\|28317 | Unnamed protein product | | 59.5/5.17 | 200 | | 4 | 7 | |
| G6-1 | gi\|189054178 | Unnamed protein product | 31.9/4.82 | 66.0/7.62 | 364 | 133 | 6 | 11 | 10.00 |
| G6-2 | gi\|6694937 | Nudix hydrolase NUDT5 | | 24.2/4.74 | 156 | | 2 | 12 | |
| G7 | gi\|7331218 | Keratin 1 | 28.3/5.10 | 66.0/8.16 | 127 | 114 | 2 | 3 | 4.26 |
| G8 | gi\|188492 | Heat shock-induced protein | 27.4/4.91 | 70.4/5.76 | 110 | 108 | 3 | 6 | 3.52 |
| G9-1 | gi\|7331218 | Keratin 1 | 27.5/5.14 | 66.0/8.16 | 144 | 99 | 3 | 4 | 1.79 |
| G9-2 | gi\|431422 | Ran/TC4 binding protein | | 23.6/5.15 | 87 | | 2 | 9 | |
| G9-3 | gi\|5729877 | Heat shock 70 kDa | | 70.8/5.37 | 51 | | 1 | 1 | |
| | | protein 8 isoform 1 | | | | | | | |
| G11 | gi\|553734 | Putative protein | 26.0/4.80 | NA | 33 | 62 | 1 | NA | 10.00 |
| G12 | gi\|4505591 | Peroxiredoxin 1 | 24.2/8.41 | 22.1/8.27 | 183 | 57 | 6 | 39 | 3.66 |
| G14 | gi\|349905 | Chain F, mutant recombinant | 19.7/5.74 | 15.7/5.70 | 66 | 41 | 3 | 18 | 1.70 |
| | | human Cu, Zn | | | | | | | |
| | | superoxide dismutase | | | | | | | |
| G15 | gi\|5031635 | Cofilin 1 (non-muscle) | 18.5/8.48 | 18.5/8.22 | 220 | 55 | 7 | 31 | 2.00 |
| lm | gi\|2981743 | Chain A, secypa | 16.7/7.61 | 17.8/7.82 | 251 | 47 | 9 | 33 | 1 |
| | | complexed Withhagpia | | | | | | | |
| | | (Pseudo-symmetric monomer) | | | | | | | |

In summation, nanogold particles can be applied in human chronic myelogenous leukemia K562 cells and/or human lymphoma cells to induce endoplasmic reticulum stress, and nanogold particles or related compositions can be used as pharmaceuticals or medical food. In view of these features, the present invention further provides a composition capable of inducing endoplasmic reticulum stress, and the composition comprises a plurality of nanogold particles and a carrier or excipient, wherein after a chronic myelogenous leukemia patient intakes the composition capable of inducing endoplasmic reticulum stress, the endoplasmic reticulum stress of the chronic myelogenous leukemia cells can be induced; and after a lymphoma patient intakes the composition capable of inducing endoplasmic reticulum stress, the endoplasmic reticulum stress of the lymphoma cells can be induced.

While the invention has been described by means of specific embodiments, numerous modifications and variations could be made thereto by those skilled in the art without departing from the scope and spirit of the invention set forth in the claims.

1. A composition capable of inducing endoplasmic reticulum stress, comprising a plurality of nanogold particles and a solvent; wherein a content of the nanogold particles is ranging from 0.5 μg to 5 μg per gram of the composition, and the nanogold particles have a particle size ranging from 1 nm to 30 nm.

2. The composition capable of inducing endoplasmic reticulum stress as recited in claim 1, wherein the solvent is selected from the group consisting of water and alcohol.

3. (canceled)

4. (canceled)

5. The composition capable of inducing endoplasmic reticulum stress as recited in claim 1, wherein the composition can induce endoplasmic reticulum stress in cells which are selected from the group consisting of: human chronic myelogenous leukemia K562 cells and human lymphoma cells.

6. A composition capable of inducing endoplasmic reticulum stress, comprising:
a plurality of nanogold particles; and
a carrier or excipient;
wherein the endoplasmic reticulum stress can be induced after a chronic myelogenous leukemia cell intakes the composition; wherein a content of the nanogold particles is ranging from 0.5 μg to 5 μg per gram of the composition, and the nanogold particles have a particle size ranging from 1 nm to 30 nm; wherein the composition can induce endoplasmic reticulum stress in cells which can be selected from the group consisting of: human chronic myelogenous leukemia K562 cells and human lymphoma cells.

7. (canceled)

8. (canceled)

9. A composition capable of inducing endoplasmic reticulum stress, comprising:
a plurality of nanogold particles; and
a carrier or excipient;
wherein the endoplasmic reticulum stress can be induced after a lymphoma cell intakes the composition; wherein a content of the nanogold particles is ranging from 0.5 μg to 5 μg per gram of the composition, and the nanogold particles have a particle size ranging from 1 nm to 30 nm; wherein the composition can induce endoplasmic reticulum stress in cells which can be selected from the group consisting of: human chronic myelogenous leukemia K562 cells and human lymphoma cells.

10. (canceled)

11. (canceled)
